(19) United States
(12) Patent Application Publication
(10) Pub. No.: US 2024/0261219 A1
(43) Pub. Date: Aug. 8, 2024

(54) NEW OIL-IN-WATER NANOEMULSION

(71) Applicant: TINY TECHNOLOGIES GMBH, Hamburg (DE)

(72) Inventors: Thorge DEBUS, Hamburg (DE); Oliver DROZDALSKI, Hamburg (DE); Tobias MELLER, Hamburg (DE)

(21) Appl. No.: 18/565,760

(22) PCT Filed: Jun. 3, 2022
(86) PCT No.: PCT/EP2022/065202

§ 371 Date: Nov. 30, 2023

(30) Foreign Application Priority Data
Jun. 3, 2021 (DE) 10 2021 205 664.4

(51) Int. Cl. A61K 9/107; A23D 7/005; A61K 8/06; A61K 31/00; A61K 47/10; A61K 47/14; A61K 47/44; A61Q 19/00

(52) U.S. Cl. CPC A61K 9/1075; A23D 7/005; A61K 8/068; A61K 31/658; A61K 47/10; A61K 47/14; A61K 47/44; A61Q 19/00

(57) ABSTRACT

The invention relates to a novel oil-in-water nanoemulsion on vegetable oil basis, which is particularly suitable for providing fat-soluble substances. The nanoemulsion consists exclusively of natural substances and is therefore particularly suitable for oral application. In addition, the nanoemulsion is characterized by high long-term stability and shelf life. The nanoemulsion can be used in the field of pharmacology or cosmetics as well as an additive in foodstuffs. A method for producing the novel oil-in-water nanoemulsion is also provided.

The invention relates to a novel oil-in-water nanoemulsion on vegetable oil basis, which is particularly suitable for providing fat-soluble substances. The nanoemulsion consists exclusively of natural substances and is therefore particularly suitable for oral application. In addition, the nanoemulsion is characterized by high long-term stability and shelf life. The nanoemulsion can be used in the field of pharmacology or cosmetics as well as an additive in foodstuffs. A method for producing the novel oil-in-water nanoemulsion is also provided.

### BACKGROUND OF THE INVENTION

Nanoemulsions are generally defined as kinetically stable colloidal systems that have a droplet size in the order of 100 nm or less. Nanoemulsions have improved functional properties compared to conventional emulsions, which makes their use attractive in many industrial fields. For example, nanoemulsions are used for the administration of lipophilic substances in pharmacology, cosmetics and the food industry.

A frequently observed problem with nanoemulsions is their low stability and their inadequate ability to deliver lipophilic substances in sufficient quantities. Emulsions that are capable of delivering high quantities of lipophilic substances often prove to be relatively unstable in practice, meaning that they can only be stored in a stable condition for a few weeks. Conversely, stable emulsions with small droplet sizes regularly have a low capacity for loading the oil phase, so that the quantities of substances to be administered are small. The production of nanoemulsions which have balanced properties, i.e., which have a high storage stability on the one hand and a high loading capacity on the other, is extremely difficult in practice. The problem to be solved by the present invention is therefore to provide a nanoemulsion with balanced properties which can be stored for a period of several months or even years and at the same time is characterized by a high loading capacity of the oil phase. The nanoemulsion should be well tolerated and less toxic so that it can be used in pharmaceutical and food applications.

### DESCRIPTION OF THE INVENTION

According to the present invention, this problem is solved by a selection of ingredients which result in nanoemulsions which are extremely stable, even on storage, and furthermore can be loaded with large amounts of lipophilic substances which are provided to a subject with a high bioavailability after administration to the subject.

In a first aspect, the present invention thus relates to an oil-in-water nanoemulsion, wherein the oil-in-water nanoemulsion comprises the following components:
(a) an oil phase comprising a vegetable oil;
(b) a continuous phase comprising water and glycerol;
(c) an emulsifier selected from the group consisting of lecithin, mono- and diglycerides of fatty acids, lactic acid and/or citric acid and mixtures thereof;
(d) ethanol;

wherein the nanoemulsion has an average particle diameter (Dm) of 70 nm or less.

The average particle diameter (Dm) is understood to be the average diameter of the oil droplets dispersed in the continuous phase. In one embodiment, the average particle diameter of the nanoemulsion is less than 70 nm, preferably less than 65 nm, less than 60 nm, less than 55 nm, less than 50 nm, less than 45 nm, or even less than 40 nm. Thus, the average particle diameter of the nanoemulsion according to the invention can be in the range of 40-70 nm, such as in the range of 45-65 nm or in the range of 50-60 nm.

The nanoemulsion of the present invention comprises an oil phase dispersed in a continuous aqueous phase. According to the invention, the oil phase comprises a vegetable oil. The vegetable oil may be an LCT vegetable oil or an MCT vegetable oil.

In a particularly preferred embodiment, the vegetable oil is an LCT vegetable oil. In the context of the present invention, LCT vegetable oils are considered to be vegetable oils which consist of 70% (w/w) or more long-chain triglycerides (LCT), based on the total weight of the vegetable oil. Long-chain triglycerides are understood to be triglycerides that are esterified with fatty acids that have a chain length of C14-C24. These fatty acids include the saturated fatty acids myristic acid (C14), palmitic acid (C16), stearic acid (C18), arachidic acid (C20), behenic acid (C22) and lignocerin acid (C24) as well as the unsaturated fatty acids palmitoleic acid (C16), oleic acid (C18), gadoleic acid (C20) and cetoleic acid (C22). Vegetable oils that consist of 70% or more long-chain triglycerides include sunflower oil, rapeseed oil, olive oil, linseed oil, maize germ oil, wheat germ oil, palm oil, hemp oil and soybean oil.

In a further embodiment, the vegetable oil is an MCT vegetable oil. In the context of the present invention, MCT vegetable oils are considered to be vegetable oils which consist of less than 70% (w/w) long-chain triglycerides, based on the total weight of the vegetable oil. In contrast to LCT vegetable oils, these vegetable oils contain a high proportion of medium-chain triglycerides. Medium-chain triglycerides are understood to be triglycerides that are esterified with fatty acids that have a chain length of C6-C12. These fatty acids include the saturated fatty acids caproic acid (C6), caprylic acid (C8), capric acid (C10) and lauric acid (C12). MCT vegetable oils which can be used for carrying out the invention usually have a content of medium-chain triglycerides of 20% (w/w) or more, preferably 30% (w/w) or more, based on the total weight of the vegetable oil. Preferred MCT vegetable oils in the context of the invention are coconut oil and palm kernel oil.

In one embodiment, the oil phase of the nanoemulsion according to the invention comprises palm oil. In one embodiment, the oil phase of the nanoemulsion according to the invention comprises sunflower oil. In a further embodiment, the oil phase of the nanoemulsion according to the invention comprises rapeseed oil. In a still further embodiment, the oil phase of the nanoemulsion according to the invention comprises olive oil. In a still further embodiment, the oil phase of the nanoemulsion according to the invention comprises linseed oil. In a still further embodiment, the oil phase of the nanoemulsion according to the invention comprises maize germ oil. In a still further embodiment, the oil phase of the nanoemulsion according to the invention comprises wheat germ oil. In a still further embodiment, the oil phase of the nanoemulsion according to the invention comprises soybean oil. In a still further embodiment, the oil phase of the nanoemulsion according to the invention comprises coconut oil. In a still further embodiment, the oil phase of the nanoemulsion according to the invention comprises palm kernel oil.

According to the invention, the vegetable oil content is between 5% and 15% (w/w), based on the total weight of the emulsion. For example, the vegetable oil may be present in an amount of more than 5% (w/w), more than 6% (w/w), more than 7% (w/w), more than 8% (w/w), more than 9% (w/w), more than 10% (w/w), more than 11% (w/w), more than 12% (w/w), more than 13% (w/w) or more than 14% (w/w). It is particularly preferred that the vegetable oil is present in an amount of 5-10% (w/w) in the emulsion, e.g., in an amount of 6-10% (w/w), 7-10% (w/w) or 8-10% (w/w).

In addition to the oil phase, the nanoemulsion according to the invention comprises a continuous phase, which comprises water and glycerol in addition to other possible components. The proportion of glycerol in the continuous phase can vary depending on the area of application of the emulsion. However, according to the invention, the proportion of glycerol is between 30% and 70% (w/w), based on the total weight of the emulsion. In one embodiment of the invention, the proportion of glycerol in the nanoemulsion according to the invention is at least 30% (w/w), at least 35% (w/w), at least 40% (w/w), at least 45% (w/w), at least 50% (w/w), at least 55% (w/w), at least 60% (w/w) or at least 65% (w/w), based on the total weight of the emulsion. Thus, the proportion of glycerol in the nanoemulsion may be 35-70% (w/w), 40-70% (w/w), 45-70% (w/w), 50-70% (w/w), 55-70% (w/w) or between 60-70% (w/w), based on the total weight of the emulsion.

The nanoemulsion according to the invention further comprises one or more emulsifiers which facilitate the preparation of the nanoemulsion and stabilize the emulsion after its preparation. The one or more emulsifiers act as surfactants active substances which reduce the interfacial tension at the oil-water phase interface. According to the invention, the one or more emulsifiers used are selected from the group consisting of lecithin and mono- and diglycerides of fatty acids, lactic acid and/or citric acid and mixtures thereof. In a preferred embodiment, the nanoemulsion comprises lecithin as emulsifier. In another preferred embodiment, the nanoemulsion comprises monoglycerides and/or diglycerides of fatty acids, lactic acid and/or citric acid or mixtures thereof as emulsifier. This means that the nanoemulsion may comprise glycerol which is single or double esterified with an acid. The acid may be a saturated or unsaturated fatty acid, lactic acid and/or citric acid. The fatty acids linoleic acid, oleic acid and stearic acid are particularly preferred.

The nanoemulsion may thus comprise one or more of the following compounds as emulsifier: glyceryl citrate, glyceryl lactate, glyceryl linoleate, glyceryl oleate (Imwitor 948), glyceryl stearate (Imwitor 491), glyceryl lactate citrate, glyceryl linoleate citrate, glyceryl stearate citrate (Imwitor 372P), glyceryl oleate citrate, glyceryl linoleate lactate, glyceryl stearate lactate, glyceryl oleate lactate, glyceryl oleate linoleate, glyceryl stearate linoleate, glyceryl oleate stearate and mixtures of the above compounds.

In a preferred embodiment, the nanoemulsion comprises lecithin as the sole emulsifier. In another preferred embodiment, the nanoemulsion comprises glyceryl citrate as the sole emulsifier. In yet another preferred embodiment, the nanoemulsion comprises glyceryl lactate as the sole emulsifier. In still another preferred embodiment, the nanoemulsion comprises glyceryl linoleate as the sole emulsifier. In yet another preferred embodiment, the nanoemulsion comprises glyceryl oleate as the sole emulsifier.

However, mixtures of these emulsifiers can also be used in an advantageous manner. In a particularly preferred embodiment, the emulsifier used in the preparation of the nanoemulsion according to the invention is a mixture of mono- and diglycerides of lactic acid, citric acid, linoleic acid and oleic acid (glyceryl citrate/lactate/linoleate/oleate). Such a mixture of different emulsifiers is marketed by several suppliers under the tradename Imwitor 375. In a particularly preferred embodiment, the emulsifier present in the nanoemulsion is therefore Imwitor 375.

In a further preferred embodiment, the emulsifier present in the nanoemulsion is thus glyceryl stearate, which is marketed by several suppliers under the name Imwitor 491. In a still further preferred embodiment, the emulsifier present in the nanoemulsion is thus glyceryl stearate citrate, which is marketed by several suppliers under the tradename Imwitor 372 P. In a particularly preferred embodiment, the emulsifier present in the nanoemulsion is thus glyceryl oleate, which is marketed by several suppliers under the name Imwitor 948.

According to the invention, it is preferred that the total amount of emulsifier in the nanoemulsion is between 2-10% (w/w), more preferably between 3-10% (w/w), 4-10% (w/w), 5-10% (w/w), 6-10% (w/w), 7-10% (w/w) or 8-10% (w/w), based on the total weight of the emulsion. Thus, the total amount of emulsifier in the nanoemulsion according to the invention is preferably more than 3% (w/w), more than 4% (w/w), more than 5% (w/w), more than 6% (w/w), more than 7% (w/w) or more than 8% (w/w). A total amount of emulsifier of 5-6% (w/w) is particularly preferred.

In one embodiment, the emulsifier in the nanoemulsion is lecithin present in a total amount between 3-10% (w/w), 4-10% (w/w), 5-10% (w/w), 6-10% (w/w), 7-10% (w/w) or 8-10% (w/w), based on the total weight of the emulsion. The total amount of lecithin in the nanoemulsion is preferably more than 3% (w/w), more than 4% (w/w), more than 5% (w/w), more than 6% (w/w), more than 7% (w/w) or more than 8% (w/w). A total amount of lecithin of 5-6% (w/w) is particularly preferred.

In another embodiment, the emulsifier in the nanoemulsion is a mixture of two or more of the compounds glyceryl citrate, glyceryl lactate, glyceryl linoleate, glyceryl oleate (Imwitor 948), glyceryl stearate (Imwitor 491), glyceryl lactate citrate, glyceryl linoleate citrate, glyceryl stearate citrate (Imwitor 372P), glyceryl oleate citrate, glyceryl linoleate lactate, glyceryl stearate lactate, glyceryl oleate lactate, glyceryl oleate linoleate, glyceryl stearate linoleate and glyceryl oleate stearate, wherein the mixture is present in a total amount of between 3-10% (w/w), 4-10% (w/w), 5-10% (w/w), 6-10% (w/w), 7-10% (w/w) or 8-10% (w/w) in the nanoemulsion, based on the total weight of the emulsion. The mixture may be present in a total amount of more than 3% (w/w), more than 4% (w/w), more than 5% (w/w), more than 6% (w/w), more than 7% (w/w) or more than 8% (w/w). A total amount of 5-6% (w/w) of the mixture in the emulsion is particularly preferred, based on the total weight of the emulsion. The mixture is preferably Imwitor 375 (glyceryl citrate/lactate/linoleate/oleate).

In another embodiment, the emulsifier in the nanoemulsion is a mixture of lecithin with one or more of glyceryl citrate, glyceryl lactate, glyceryl linoleate, glyceryl oleate (Imwitor 948), glyceryl stearate (Imwitor 491), glyceryl lactate citrate, glyceryl linoleate citrate, glyceryl stearate citrate (Imwitor 372P), glyceryl oleate citrate, glyceryl linoleate lactate, glyceryl stearate lactate, glyceryl oleate lactate, glyceryl oleate linoleate, glyceryl stearate linoleate and glyceryl oleate stearate, wherein the mixture is present in a total amount between 3-10% (w/w), 4-10% (w/w), 5-10% (w/w), 6-10% (w/w), 7-10% (w/w) or 8-10% (w/w) in the nanoemulsion, based on the total weight of the emulsion. The mixture of lecithin with one or more of the above glycerol esters may be present in a total amount of more than 3% (w/w), more than 4% (w/w), more than 5% (w/w), more than 6% (w/w), more than 7% (w/w) or more than 8% (w/w). A total amount of 5-6% (w/w) of the mixture in the emulsion is particularly preferred, based on the total weight of the emulsion. The mixture is preferably a mixture of lecithin and Imwitor 375 (glyceryl citrate/lactate/linoleate/oleate).

As a further component, the nanoemulsion according to the invention comprises ethanol. The ethanol is preferably present in the nanoemulsion in an amount of 2-20% (w/w). For example, the ethanol may be present in an amount of more than 2% (w/w), more than 3% (w/w), more than 4% (w/w), more than 5% (w/w), more than 6% (w/w), more than 7% (w/w), more than 8% (w/w), more than 9% (w/w), more than 10% (w/w), more than 11% (w/w), more than 12% (w/w), more than 13% (w/w), more than 14% (w/w), more than 15% (w/w), more than 16% (w/w), more than 17% (w/w), more than 18% (w/w) or more than 19% (w/w). It is particularly preferred that the ethanol is present in the emulsion in an amount of 5-10% (w/w), e.g. in an amount of 6-10% (w/w), in an amount of 7-10% (w/w) or in an amount of 8-10% (w/w).

In one embodiment, the oil phase of the nanoemulsion according to the invention comprises a poorly water-soluble or water-insoluble lipophilic compound which is to be released after uptake of the nanoemulsion. The poorly water-soluble or water-insoluble lipophilic compound is preferably selected from the group consisting of cannabidiol (CBD), cannabigerol (CBG), cannabichromene (CBC), cannabinol (CBN), tetrahydrocannabinol (THC), melatonin, resveratrol, astaxanthin, coenzyme Q10, vitamin A, vitamin C, vitamin E, vitamin D3, vitamin K2, flavonoids, glutathione, B-caryophyllene, 2-arachidonylglycerol (2-AG), palmitoylethanolamide (PEA), anandamide, oleoylethanolamide (OEA) and oligomeric proanthocyanidines (OPC). In a particularly preferred embodiment, the oil phase of the nanoemulsion according to the invention comprises cannabidiol (CBD), which is released after ingestion or administration of the nanoemulsion to a subject or patient.

In addition to the components described in detail above, the nanoemulsion according to the invention may comprise further optional components, depending on the field of application of the respective emulsion. Thus, in certain embodiments, it is advantageous if the oil phase comprises an essential oil. On the one hand, this component can serve as a preservative or antioxidant. On the other hand, the essential oil can influence the taste of a nanoemulsion intended for oral administration as well as the dissolution behavior of the other components. Suitable essential oils include, for example, orange peel oil, lemon oil and peppermint oil. According to the invention, the essential oil is used in an amount of 0.5-5% (w/w),

In addition, the nanoemulsion according to the invention may comprise substances which increase the bioavailability of the lipophilic compound to be administered. According to a particularly preferred embodiment, suitable substances for increasing bioavailability are selected from the group consisting of piperine, curcumin, resveratrol, quercetin, menthol, naringin, bergamottin, kaempferol and rutin.

The oil phase of the nanoemulsion according to the invention may further comprise oleic acid and/or ethyl oleate. Oleic acid and ethyl oleate can act as co-emulsifiers or solvents in the nanoemulsion. They can also prevent and/or delay the ripening of the nanoemulsion. Oleic acid and ethyl oleate may be present in the nanoemulsion in a total amount of 0.5-10% (w/w). In addition, the nanoemulsion according to the invention may comprise one or more sugar esters. The sugar ester may be a sucrose ester. Suitable sucrose esters suitable as additives for the nanoemulsions according to the invention include, but are not limited to, sucrose stearate, sucrose palmitates, sucrose myristate and/or sucrose laurates. The one or more sugar esters may be present in a total amount of 0.5-3% (w/w) in the nanoemulsion. In a particularly preferred embodiment, the sucrose ester is sucrose stearate of the following structure:

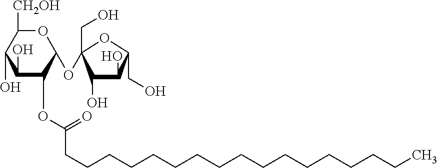

The nanoemulsion according to the invention has the particular advantage that only purely natural compounds can be used in its production. Thus, according to the invention, nanoemulsions with the special, advantageous properties can also be produced without having to resort to ethoxylated compounds. In a preferred embodiment, the nanoemulsion according to the invention contains only a minimal amount of ethoxylated compounds, such as ethoxylated glyceryl fatty acid esters or sorbitan fatty acid esters, such as polysorbate 80. In a particularly preferred embodiment, the total amount of ethoxylated compounds in the nanoemulsion according to the invention is less than 0.5% (w/w), more preferably less than 0.4% (w/w), less than 0.3% (w/w), less than 0.2% (w/w), less than 0.1% (w/w), less than 0.09% (w/w), less than 0.08% (w/w), less than 0.07% (w/w), less than 0.06% (w/w), less than 0.05% (w/w), less than 0.04% (w/w), less than 0.03% (w/w), less than 0.02% (w/w), less than 0.01% (w/w), less than 0.005% (w/w) or less than 0.001% (w/w). According to the invention, it is particularly preferred that the nanoemulsion described herein does not contain any ethoxylated compounds, such as ethoxylated glyceryl fatty acid esters or sorbitan fatty acid esters.

The nanoemulsion of the present invention is characterized by a particularly high shelf life. This means that the average particle size of the nanoemulsion does not change significantly even after prolonged storage. According to the invention, it is preferred that the average particle size of the nanoemulsion does not exceed a size of 100 nm after storage for 2 months, more preferably 4 months, even more preferably 6 months. The storage preferably takes place at a temperature range of 20-24° C.

In one embodiment, the nanoemulsion according to the invention has an average particle size of 100 nm or less, and preferably 80 nm or less, after 2 months of storage at a temperature of 20-24° C. In a further embodiment, the nanoemulsion according to the invention has an average particle size of 100 nm or less, and preferably 80 nm or less, after 4 months of storage at a temperature of 20-24° C. In a still further embodiment, the nanoemulsion according to the invention has an average particle size of 100 nm or less, and preferably 80 nm or less, after 6 months of storage at a temperature of 20-24° C.

Particularly preferred nanoemulsions of the present invention are exemplified below. Thus, in one embodiment, the present invention relates to an oil-in-water nanoemulsion comprising the following components:
(a) an oil phase comprising a vegetable oil, which vegetable oil comprises medium-chain and/or long-chain triglycerides, wherein the vegetable oil is present in an amount of 5-15% (w/w), based on the total weight of the emulsion;
(b) a continuous phase comprising water and glycerol, wherein glycerol is present in an amount of 30-70% (w/w), based on the total weight of the emulsion;
(c) an emulsifier selected from the group consisting of lecithin, mono- and diglycerides of fatty acids, lactic acid and/or citric acid and mixtures thereof, wherein the emulsifier is present in an amount of 2-10% (w/w), based on the total weight of the emulsion;
(d) ethanol, which is present in an amount of 2-20% (w/w), based on the total weight of the emulsion;

wherein the nanoemulsion has an average particle diameter (Dm) of 70 nm or less.

In another embodiment, the present invention relates to an oil-in-water nanoemulsion comprising the following components:
(a) an oil phase comprising a vegetable oil, which vegetable oil comprises medium-chain and/or long-chain triglycerides, wherein the vegetable oil is present in an amount of 5-8% (w/w), based on the total weight of the emulsion;
(b) a continuous phase comprising water and glycerol, wherein glycerol is present in an amount of 50-70% (w/w), based on the total weight of the emulsion;
(c) an emulsifier selected from the group consisting of lecithin, mono- and diglycerides of fatty acids, lactic acid and/or citric acid and mixtures thereof, wherein the emulsifier is present in an amount of 5-8% (w/w), based on the total weight of the emulsion;
(d) ethanol, which is present in an amount of 5-10% (w/w), based on the total weight of the emulsion;

wherein the nanoemulsion has an average particle diameter (Dm) of 70 nm or less.

In another embodiment, the present invention relates to an oil-in-water nanoemulsion comprising the following components:
(a) an oil phase comprising one or more vegetable oils selected from the group consisting of palm oil, sunflower oil, olive oil and soybean oil;
(b) a continuous phase comprising water and glycerol, wherein glycerol is present in an amount of 50-70% (w/w), based on the total weight of the emulsion;
(c) an emulsifier, wherein the emulsifier is lecithin, which is present in an amount of 5-8% (w/w), based on the total weight of the emulsion;
(d) ethanol, which is present in an amount of 5-10% (w/w), based on the total weight of the emulsion;

wherein the nanoemulsion has an average particle diameter (Dm) of 70 nm or less.

In a further aspect, the present invention relates to a method of preparing a nanoemulsion as described above, the method comprising the steps of
(a) premixing glycerol, water, ethanol and one or more emulsifiers selected from the group consisting of lecithin, mono- and diglycerides of fatty acids, lactic acid and/or citric acid and mixtures thereof;
(b) adding a vegetable oil comprising predominantly medium-chain or long-chain triglycerides; and
(c) subjecting the mixture to high-pressure homogenization at a pressure of 800-1500 bar.

In step (a) of the method according to the invention, glycerol, water, ethanol and one or more emulsifiers selected from the group consisting of lecithin, mono- and diglycerides of fatty acids, lactic acid and/or citric acid and mixtures thereof are mixed together. The individual ingredients can be added in the respective amounts while stirring in a suitable vessel, such as a glass container or glass flask.

In step (b) of the method according to the invention, a vegetable oil comprising medium-chain or long-chain triglycerides is added. Vegetable oils suitable for the preparation of the nanoemulsions according to the invention have been described above. In one embodiment, the vegetable oil added to the previously prepared mixture is a vegetable oil comprising more than 50% medium chain triglycerides, such as coconut oil or palm kernel oil. In another embodiment, the vegetable oil which is added to the previously prepared mixture is a vegetable oil which consists of more than 50% long-chain triglycerides, such as sunflower oil, rapeseed oil, olive oil, linseed oil, maize germ oil, wheat germ oil or soybean oil.

In one embodiment, the oil phase of the nanoemulsion according to the invention comprises palm oil. In another embodiment, the oil phase of the nanoemulsion according to the invention comprises sunflower oil. In a further embodiment, the oil phase of the nanoemulsion according to the invention comprises hemp oil. In a further embodiment, the oil phase of the nanoemulsion according to the invention comprises rapeseed oil. In a still further embodiment, the oil phase of the nanoemulsion according to the invention comprises olive oil. In a still further embodiment, the oil phase of the nanoemulsion according to the invention comprises linseed oil. In a still further embodiment, the oil phase of the nanoemulsion according to the invention comprises maize germ oil. In a still further embodiment, the oil phase of the nanoemulsion according to the invention comprises wheat germ oil. In a still further embodiment, the oil phase of the nanoemulsion according to the invention comprises soybean oil. In a still further embodiment, the oil phase of the nanoemulsion according to the invention comprises coconut oil. In a still further embodiment, the oil phase of the nanoemulsion according to the invention comprises palm kernel oil.

In step (c) of the method according to the invention, the mixture obtained from the previous step (b) is subjected to high pressure homogenization at a pressure of 800-1500 bar in order to reduce the size of the oil droplets in the continuous phase.

This step is preferably carried out in a device suitable for high-pressure homogenization. Devices for high pressure homogenization are sufficiently well known in the prior art and can be obtained from several commercial suppliers. Suitable devices include, for example, the LM10 Microfluidizer or the M-110L Microfluidizer (Microfluidics, Westwood, USA). Microfluidization devices reduce the average size of oil droplets in the continuous phase by passing the initial emulsion at high pressure and high velocity through a chamber with geometrically defined channels. The chamber of the microfluidization device may comprise a plurality of geometrically defined channels, such as 2, 3, 4, 5, 6, 7, 8, 9, 10 or more channels. The chamber of the microfluidization device is preferably made of ceramic or stainless steel.

In the context of the present invention, high-pressure homogenization, e.g., microfluidization, is carried out at a pressure between 800-1500 bar. According to the invention, it is particularly preferred that the high-pressure homogenization, e.g. the microfluidization, is carried out at a pressure of at least 800 bar, more preferably at least 850 bar, at least 900 bar, at least 950 bar, at least 1000 bar, at least 1050 bar, at least 1100 bar, at least 1150 bar, at least 1200 bar, at least 1250 bar, at least 1300 bar, at least 1350 bar, at least 1400 bar, or at least 1450 bar.

To achieve the small particle size, it may be advantageous to cycle the mixture obtained from step (b) through the high-pressure homogenization device several times, since the particle size in the continuous phase is further reduced with each cycle. In the method, it is preferred to cycle the mixture obtained from step (b) 2-15 times, preferably 2-10 times, through the high-pressure homogenization device. Thus, the mixture obtained from step (b) can preferably be cycled through the high-pressure homogenization device at least 2 times, at least 3 times, at least 4 times, at least 5 times, at least 6 times, at least 7 times, at least 8 times, at least 9 times or at least 10 times.

The nanoemulsions according to the invention can be used in many fields. For example, they can be used in the field of medicine or pharmacology to administer lipophilic pharmaceutical agents. In a further aspect, the present invention thus relates to a nanoemulsion as described above for use in medicine or pharmacology. The nanoemulsion may be formulated for these purposes for various forms of administration. However, it is preferred that the nanoemulsion is formulated for oral administration.

In a still further aspect, the present invention thus relates to the use of a nanoemulsion as described above for the preparation of a pharmaceutical or cosmetic composition. Preferably, the pharmaceutical or cosmetic composition comprises a lipophilic compound which is pharmaceutically or cosmetically active, such as cannabidiol (CBD). The invention also relates to a nanoemulsion as described above for the preparation of a food or feed product, such as a beverage.

The invention therefore also relates to the use of a nanoemulsion as described above as an additive in foodstuffs or in cosmetics. Since the nanoemulsion can penetrate the upper skin layers when applied topically and reach deeper skin layers, it is particularly suitable for the delivery of lipophilic cosmetic or pharmaceutical active ingredients to deeper skin layers.

In a further aspect, the present invention relates to a nanoemulsion as described above for use in a therapeutic method in which a lipophilic pharmaceutically active compound is administered. In a still further aspect, the present invention relates to a nanoemulsion as described above for providing a lipophilic compound to a subject in need thereof. Preferably, the lipophilic compound is cannabidiol (CBD).

### DESCRIPTION OF THE FIGURES

FIG. 1 shows the results of the particle size determination of the nanoemulsion 1 produced in example 1.

FIG. 2 shows the results of the particle size determination of the nanoemulsion 2 produced in example 1.

FIG. 3 shows the results of the particle size determination of the nanoemulsion 3 produced in example 1.

FIG. 4 shows the results of the particle size determination of the nanoemulsion 4 produced in Example 1.

FIG. 5 shows the results of the determination of the long-term stability of the nanoemulsion 1 produced in Example 1.

FIG. 6 shows the polydispersity index (PDI) data of the nanoemulsion 1 produced in Example 1.

### EXAMPLES

The following examples are intended to illustrate the present invention. However, they are not to be understood as limiting the scope of the invention.

### Example 1: Production of Nanoemulsions

Nanoemulsion 1: To prepare a first nanoemulsion, a pre-emulsion was prepared from lecithin P75 (Lipoid GmbH), glycerol (99%, Gustav Heess GmbH), ethanol (96%, Sigma-Aldrich) and unrefined red palm oil (Gustav Heess GmbH). For this purpose, 6% (w/w) lecithin was dissolved at 40° C. under constant stirring in a mixture of 60% (w/w) glycerol, 16% (w/w) demineralized water and 10% (w/w) ethanol. After 3 hours, 8% (w/w) unrefined red palm oil was added as oil phase. The pre-emulsion was obtained by stirring with a VISCO-JET at 800 rpm and 40° C. for two hours. The pre-emulsion was then passed through a microfluidizer (LM10 Microfluidizer, Microfluidics International Corporation, Canada) 12 times at 1400 bar. After each pass, the mixture was cooled to below 20° C.

Nanoemulsion 2: To prepare a second nanoemulsion, a pre-emulsion was first prepared from Imwitor 375 (101 Oleo GmbH), glycerol (99%, Gustav Heess GmbH), ethanol (96%, Sigma-Aldrich) and unrefined red palm oil (Gustav Heess GmbH). For this purpose, 6% (w/w) Imwitor 375 was dissolved at 40° C. under constant stirring in a mixture of 60% (w/w) glycerol, 16% (w/w) demineralized water and 10% (w/w) ethanol. After 3 hours, 8% (w/w) unrefined red palm oil was added as oil phase. The pre-emulsion was obtained by stirring with a VISCO-JET at 800 rpm and 40° C. for two hours. The pre-emulsion was then passed through a microfluidizer (LM10 Microfluidizer, Microfluidics International Corporation, Canada) 12 times at 1400 bar. After each pass, the mixture was cooled to below 20° C.

Nanoemulsion 3: To prepare a third nanoemulsion, a pre-emulsion was prepared from lecithin P75 (Lipoid GmbH), glycerol (99%, Gustav Heess GmbH), ethanol (96%, Sigma-Aldrich), sugar ester (Sisterna SP70-C, Sisterna) and unrefined red palm oil (Gustav Heess GmbH).

For this purpose, 5.5% (w/w) lecithin and 0.5% (w/w) sugar ester were dissolved at 40° C. under constant stirring in a mixture of 60% (w/w) glycerol, 16% (w/w) demineralized water and 10% (w/w) ethanol. After 3 hours, 8% (w/w) unrefined red palm oil was added as oil phase. The pre-emulsion was obtained by stirring with a VISCO-JET at 800 rpm and 40° C. for two hours. The pre-emulsion was then passed through a microfluidizer (LM10 Microfluidizer, Microfluidics International Corporation, Canada) 12 times at 1400 bar. After each pass, the mixture was cooled to below 20° C.

Nanoemulsion 4: To prepare a fourth nanoemulsion, a pre-emulsion was first prepared from Imwitor 375 (101 Oleo GmbH), glycerol (99%, Gustav Heess GmbH), ethanol (96%, Sigma-Aldrich), sugar ester (Sisterna SP70-C, Sisterna) and unrefined red palm oil (Gustav Heess GmbH). For this purpose, 5.5% (w/w) Imwitor 375 and 0.5% (w/w) sugar ester were dissolved at 40° C. under constant stirring in a mixture of 60% (w/w) glycerol, 16% (w/w) demineralized water and 10% (w/w) ethanol. After 3 hours, 8% (w/w) unrefined red palm oil was added as oil phase. The pre-emulsion was obtained by stirring with a VISCO-JET at 800 rpm and 40° C. for two hours. The pre-emulsion was then passed through a microfluidizer (LM10 Microfluidizer, Microfluidics International Corporation, Canada) 12 times at 1400 bar. After each pass, the mixture was cooled to below 20° C.

### Example 2: Determining the Particle Size

The particle size of the nanoemulsions produced in example 1 was determined by dynamic light scattering (DLS, Malvern Nano ZS90, Malvern, UK). The samples (1 ml) were dispersed in 300 ml demineralized water. DLS measurements were performed at 25° C. and 173° scattering angle.

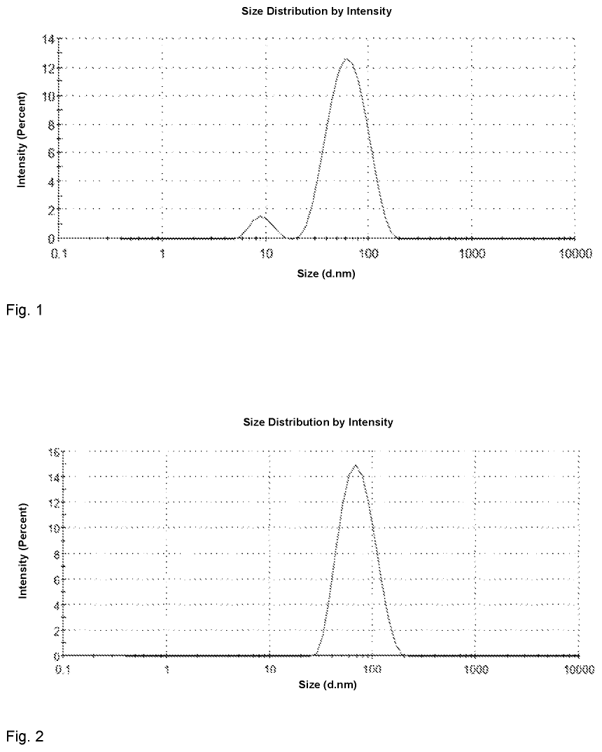

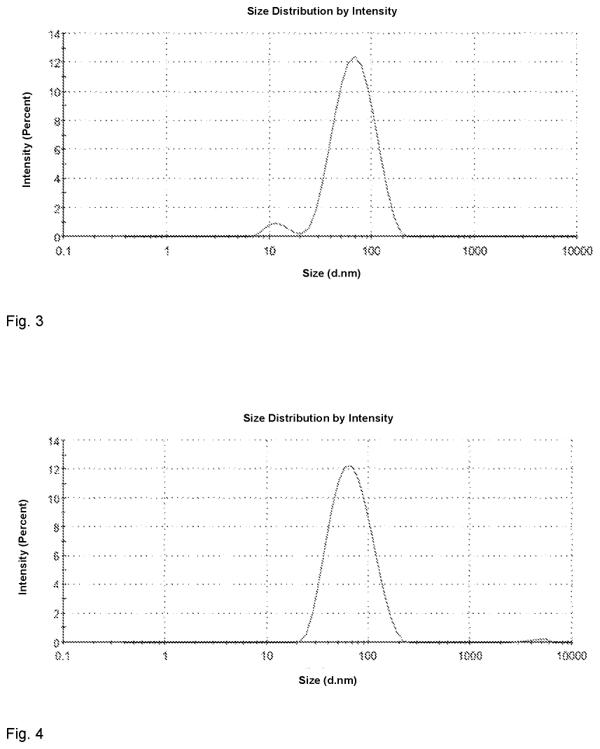

The result is shown in FIGS. 1-4. As can be seen from the figures, the average particle diameter of the nanoemulsion 1 was approximately 47.8 nm. The average particle diameter of nanoemulsion 2 was about 65.7 nm. The average particle diameter of nanoemulsion 3 was approximately 53.2 nm. The average particle diameter of nanoemulsion 4 was approximately 61.5 nm.

### Example 3: Determination of Long-Term Stability

Samples of nanoemulsion 1 produced in example 1 were stored at room temperature and 4° C. in the absence of light. Samples of the nanoemulsions were taken at intervals of one month and characterized by means of DLS measurements.

The result is shown in FIG. 5. It was found that even storage for 8 months did not significantly change the average particle diameter, which indicates the storage stability of the emulsions.

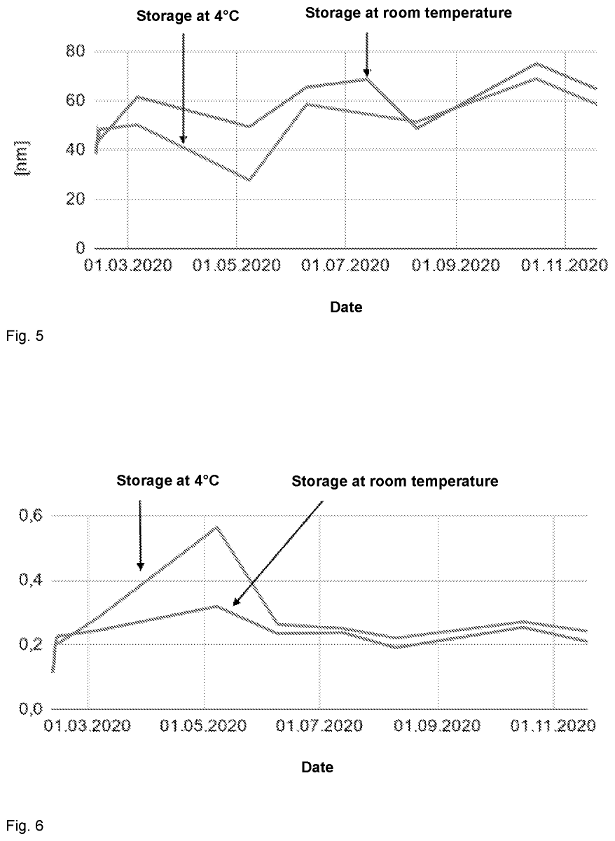

The data for the polydispersity index (PDI) calculated from the results of the DLS measurements are shown in FIG. 6.

1. Oil-in-water nanoemulsion comprising
(a) an oil phase comprising a vegetable oil, preferably an LCT vegetable oil;
(b) a continuous phase comprising water and glycerol;
(c) an emulsifier selected from the group consisting of lecithin, mono- and diglycerides of fatty acids, lactic acid and/or citric acid and mixtures thereof;
(d) ethanol;
wherein the nanoemulsion has an average particle diameter (Dm) of 70 nm or less.

2. The nanoemulsion according to claim 1, wherein the emulsifier is a mixture of mono- and diglycerides of lactic acid, citric acid, linoleic acid and oleic acid and is preferably present in an amount of 2-10% (w/w), more preferably 5-6% (w/w).

3. The nanoemulsion according to claim 1, wherein the emulsifier is lecithin and is preferably present in an amount of 2-10% (w/w), more preferably 5-6% (w/w).

4. The nanoemulsion according to claim 1, wherein the oil phase further comprises oleic acid and/or ethyl oleate.

5. The nanoemulsion according to claim 1, wherein the oil phase further comprises an essential oil.

6. The nanoemulsion according to claim 1, wherein the continuous phase comprises 30-70% (w/w) glycerol.

7. The nanoemulsion according to claim 1, wherein the ethanol is present in the nanoemulsion in an amount of 2-20% (w/w), and preferably 5-10% (w/w).

8. The nanoemulsion according to claim 1, further comprising a bioavailability enhancer selected from the group consisting of piperine, curcumin, resveratrol, quercetin, menthol, naringin, bergamottin, kaempferol and rutin.

9. The nanoemulsion according to claim 1, wherein the nanoemulsion does not contain ethoxylated compounds.

10. The nanoemulsion according to claim 1, wherein the average particle size of the nanoemulsion does not exceed 100 nm after storage for 6 months.

11. The nanoemulsion according to claim 1, further comprising a sugar ester, preferably a sucrose ester.

12. The nanoemulsion according to claim 1 for use in medicine.

13. The nanoemulsion according to claim 1 for use in a therapeutic method in which a lipophilic pharmaceutically active agent is administered.

14. Use of a nanoemulsion according to claim 1 for the preparation of a pharmaceutical or cosmetic composition.

15. Use of a nanoemulsion according to claim 1 as an additive in foodstuffs.

16. Use of a nanoemulsion according to claim 1 as an additive in cosmetics.

17. The nanoemulsion according to claim 1, wherein the nanoemulsion comprises cannabidiol (CBD).

18. Method for preparing a nanoemulsion according to claim 1 which comprises
(a) premixing glycerol, water, ethanol and an emulsifier selected from the group consisting of lecithin, mono- and diglycerides of fatty acids, lactic acid and/or citric acid and mixtures thereof;
(b) adding a vegetable oil, preferably an LCT vegetable oil; and
(c) subjecting the mixture to high-pressure homogenization at a pressure of 800-1500 bar.
